(19) United States
(12) Patent Application Publication
(10) Pub. No.: US 2007/0037990 A1
(43) Pub. Date: Feb. 15, 2007

(54) Method of producing an organic compound comprising at least one oxygenated functional group

(76) Inventors: Olivier Buyle, Autre-Eglise (BE); Veronique Mathieu, Wavre (BE); Karol Lorent, Courcelles (BE)

Correspondence Address: CONNOLLY BOVE LODGE & HUTZ, LLP, P O BOX 2207, WILMINGTON, DE, 19899, US

(21) Appl. No.: 10/558,771

(22) PCT Filed: Jun. 2, 2004
(86) PCT No.: PCT/EP04/50990

§ 371 Date: May 1, 2006

(30) Foreign Application Priority Data
Jun. 3, 2003 (FR) 03.06718

(51) Int. Cl. C07D 301/03; C07D 313/04

(52) U.S. Cl. 549/523; 549/272

(57) ABSTRACT

Process for manufacturing an organic compound comprising at least one oxygenated functional group, according to which an organic precursor comprising an oxidizable functionality is subjected to a reaction with an imidoaromatic percarboxylic acid.

The present invention relates to a process for manufacturing an organic compound comprising at least one oxygenated functional group.

Compounds containing at least one oxygenated functional group may be used, for example, as monomers, as constituents of cosmetic compositions or as surfactants.

It is known practice to perform alkene epoxidation reactions with peracetic acid as oxidizing agent. This oxidizing agent presents risks of hydrolysis for sensitive substrates and does not always give satisfactory selectivity and oxidation yield.

The invention is directed towards overcoming the problems mentioned above.

Consequently, the invention relates to a process for manufacturing an organic compound comprising at least one oxygenated functional group, according to which an organic precursor comprising an oxidizable functionality is subjected to a reaction with an imidoaromatic percarboxylic acid.

In the process according to the invention, the imidoaromatic percarboxylic acid often corresponds to the formula 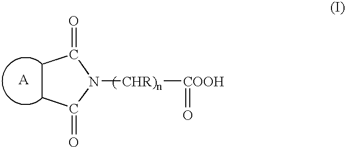
in which A represents an optionally substituted benzene or naphthalene ring, the group(s) R, which may be identical or different, represent(s) hydrogen, an optionally substituted alkyl group, COOH or COOOH and n is an integer from 1 to 5.

Preferably, all the groups R are H. n is preferably 4 or 5. ε-Phthalimido-peroxyhexanoic acid (PAP), which is commercially available from Solvay Solexis, is most particularly preferred. It has particular advantages such as being solid at room temperature and having good stability, while at the same time having sufficient reactivity in the reaction and particularly good solubility in organic synthesis media.

In the process according to the invention, the reaction is generally performed at a temperature of at least −100° C. This temperature is often at least 0° C. Preferably, it is at least 20° C. In the process according to the invention, the reaction is generally performed at a temperature of not more than 150° C. This temperature is often not more than 80° C. It is preferably not more than 60° C.

In the process according to the invention, the reaction is generally performed at a pressure of at least 0.5 bar. This pressure is preferably greater than or equal to about 1 bar (atmospheric pressure). In the process according to the invention, the reaction is generally performed at a pressure of not more than 100 bar. This pressure is often not more than 25 bar. It is preferably not more than 15 bar.

In the process according to the invention, the reaction is generally performed in a reaction medium having a content of imidoaromatic percarboxylic acid of at least 0.1% by weight relative to the total weight of the reaction medium. This content is often at least 5%. It is preferably at least 7%. In the process according to the invention, this content is generally not more than 90%. This content is often not more than 80%. It is preferably not more than 70%.

In the process according to the invention, the reaction is generally performed in a reaction medium with a content of organic precursor of at least 1% by weight relative to the total weight of the reaction medium. This content is often at least 10%. It is preferably at least 30%. In the process according to the invention, this content is generally not more than 80%. This content is often not more than 75%. It is preferably not more than 70%.

The process according to the invention may be performed in the presence of an organic solvent, preferably a polar organic solvent, for instance dioxane or tetrahydrofuran.

The process according to the invention may also be performed in the absence of solvent, the imidoaromatic percarboxylic acid generally being soluble in the organic precursor. This makes it possible to perform the reaction with a smaller volume of reaction medium and high volume productivity.

In a first embodiment, gradual addition of the imidoaromatic percarboxylic acid to the reaction medium is performed.

In a first embodiment, continuous addition of the imidoaromatic percarboxylic acid to the reaction medium is performed.

In the process according to the invention, the imidoaromatic percarboxylic acid may be introduced into the reaction in the form, for example, of a solution in an organic solvent or in pure form.

In the process according to the invention, the reaction is generally performed for a time of at least 10 minutes. This time is often at least 30 minutes. It is preferably at least 1 hour. In the process according to the invention, the reaction is generally performed for a time of not more than 50 hours. This time is often not more than 20 hours. It is preferably not more than 10 hours. A time of not more than 5 hours is more particularly preferred. It has been found that the imidoaromatic percarboxylic acid is capable of reacting rapidly with the precursor.

In one preferred variant of the process according to the invention, the reaction is performed in the substantial absence of water.

In this variant, the reaction is generally performed in a reaction medium with a water content of not more than 10% by weight relative to the total weight of the reaction medium. This content is often not more than 5%. It is preferably not more than 1%. In this variant, a reaction medium with a water content of greater than or equal to about 100 mg/kg gives good results.

After the reaction, the oxygenated compound may be separated from the reaction medium via techniques that are known per se, such as distillation or crystallization. The imidoaromatic carboxylic acid obtained as reaction by-product may be optionally removed from the reaction medium, for example via aqueous extraction.

In the process according to the invention, the organic precursor is often chosen from substituted or unsubstituted cyclic or acyclic olefins, substituted or unsubstituted cyclic or acyclic ketones, compounds containing at least one oxidizable sulfur atom, compounds containing at least one oxidizable selenium atom, compounds containing at least one oxidizable nitrogen atom, compounds containing at least one oxidizable phosphorus atom and compounds containing at least one oxidizable iodine atom.

Preferably, the organic precursor is an oxidizable fluoro compound.

When the organic precursor is an acyclic olefin, it generally contains from 2 to 100 and preferably from 2 to 20 carbon atoms. It often comprises a double bond in position 1 or 2. Preferably, it comprises a double bond in position 1 (terminal). Specific examples of acyclic olefins are ethylene, propylene, 1-butene and 2-butene, and substituted derivatives thereof, such as allyl chloride, allyl alcohol, vinyl chloride, vinylidene chloride, vinyl fluoride, vinylidene fluoride and hexafluoropropylene.

When the organic precursor is a cyclic olefin, it generally contains from 3 to 100 and preferably from 4 to 20 carbon atoms. It often comprises a ring containing from 4 to 8 and preferably 5, 6 or 7 atoms. The ring may be a carbocycle or a heterocycle. Specific examples of cyclic olefins are cyclopropene, cyclobutene, cyclopentene, cyclohexene, norbornene, dicyclopentadiene and α-pinene, and substituted derivatives thereof.

When the organic precursor is an acyclic ketone, it generally contains from 3 to 100 and preferably from 3 to 20 carbon atoms. Specific examples of acyclic ketones are acetone, butanone, pentanone and hexanone, and substituted derivatives thereof, for instance trifluoroacetone, hexafluoroacetone or 4,4′-difluorobenzophenone.

When the organic precursor is a cyclic ketone, it generally contains from 4 to 100 and preferably from 4 to 20 carbon atoms. Specific examples of cyclic ketones are cyclobutanone, cyclopentanone, cyclohexanone and cycloheptanone, and substituted derivatives thereof.

When the organic precursor is chosen from compounds containing at least one oxidizable sulfur atom, it is often chosen from symmetrical or asymmetrical thioethers and mercaptans, in particular alkyl or aryl thioethers and mercaptans, generally containing from 1 to 100 and preferably from 1 to 20 carbon atoms. Specific examples are methyl mercaptan, ethyl mercaptan, propyl mercaptan, n-butyl mercaptan, dimethyl sulfide, diethyl sulfide, dipropyl sulfide, di-n-butyl sulfide, methylethyl sulfide, methyl-n-butyl sulfide and phenylthiol, and substituted derivatives thereof.

The process according to the invention allows access to a variety of sulfur-containing organic compounds, which may, where appropriate, also serve as organic precursors, for instance disulfides, sulfenic or sulfinic acids, and disulfoxides.

When the organic precursor is chosen from compounds containing at least one oxidizable nitrogen atom, it is often chosen from amines, in particular alkyl or aryl amines and imines, generally containing from 1 to 100 and preferably from 1 to 20 carbon atoms. Primary, secondary or tertiary amines may be used. Specific examples are methylamine, dimethylamine, triethylamine, diisopropylamine, diisopropylethylamine, N,N-dimethyldodecylamine, aniline, pyridine and PhSO₂—N═CH—Ph, and substituted derivatives thereof.

The process according to the invention allows access to a variety of nitrogen-containing organic compounds, which may, where appropriate, also serve as organic precursors, for instance hydroxylamines and oximes.

When the organic precursor is chosen from compounds containing at least one oxidizable iodine atom, it is often chosen from aryl or heteroaryl iodides generally containing from 5 to 100 and preferably from 6 to 24 carbon atoms, and substituted derivatives thereof. A specific example is iodobenzene.

When the organic precursor is chosen from compounds containing at least one oxidizable phosphorus atom, it is often chosen from phosphines, in particular alkylphosphines or arylphosphines, phosphine oxides and phosphonates, generally containing from 3 to 100 and preferably from 3 to 20 carbon atoms, and substituted derivatives thereof. Specific examples are trialkylphosphines and triarylphosphines.

The examples below are intended to illustrate the invention without, however, limiting it.

### EXAMPLE 1

A solution of peracid was prepared beforehand by dissolving 277 g of PAP in 700 ml of dioxane contained in a one litre round-bottomed volumetric flask.

3.5 g of cyclohexene (0.043 mol) were introduced into a jacketed glass reactor (working volume of 70 cm³) on which is mounted a condenser cooled to −15° C. 32 ml of the peroxide solution prepared above were added gradually using a perfuser equipped with a 50 ml syringe, at a flow rate of 0.16 ml/minute, which represents 8.86 g of PAP (0.032 mol). The temperature was 60° C. The reaction medium was stirred using a magnetic bar.

After the addition of the peracid, the crude reaction product was cooled to room temperature and analysed by GC. The epoxide yield was 81%.

### EXAMPLE 2

By following procedures similar to those of Example 1, but replacing the dioxane with tetrahydrofuran, glycidol was obtained starting with allyl alcohol. The glycidol was hydrolysed to glycerol in the presence of sulfuric acid.

### EXAMPLE 3

The synthesis of epichlorohydrin by epoxidation of allyl chloride was performed in the presence of solid PAP.

The synthesis was performed in a 25 cm³ round-bottomed flask heated to 60° C. using an oil bath, and on which is mounted a condenser cooled to −10° C. by circulation of a water/ethylene glycol mixture. No stirring system was used.

1.28 g of PAP (0.0046 mol) were introduced into 2.32 g of allyl chloride (0.030 mol). The temperature was maintained at 60° C. for 17 hours. The epichlorohydrin yield was 91%.

### EXAMPLE 4

The synthesis of ε-caprolactone by oxidation of cyclohexanone was performed in the presence of solid PAP.

1.55 g of PAP (0.0056 mol) were introduced into 4.14 g of cyclohexanone (0.042 mol) in a 25 cm³ round-bottomed flask heated to 60° C. using an oil bath, and on which is mounted a condenser cooled to −10° C. by circulation of a water/ethylene glycol mixture. No stirring system was used. The temperature was maintained for 3 hours.

The ε-caprolactone yield was 75%.

### EXAMPLE 5

The synthesis of dibutyl sulfone via oxidation of dibutyl sulfide was performed in the presence of PAP dissolved in dioxane (see Example 1). The synthesis was performed in a 100 cm³ three-necked round-bottomed flask maintained at 20° C. by immersion in a thermostatically maintained water bath. A condenser cooled to −10° C. by circulation of a water/ethylene glycol mixture was mounted on the flask. A CaCl₂ cartridge was placed at the condenser outlet to prevent the entry of moisture. The reaction medium was homogenized by magnetic stirring.

2.57 g of PAP (0.0093 mol) and 21.53 g of dioxane (0.245 mol) were initially introduced into the reactor and stirred for 1 hour, followed by the introduction of dibutyl sulfide. 4.96 g (0.034 mol) of dibutyl sulfide were then added over 111 minutes.

The dibutyl sulfone yield, determined by GC, was 77%.

1. Process for manufacturing an organic compound comprising at least one oxygenated functional group, according to which an organic precursor comprising an oxidizable functionality is subjected to a reaction with an imidoaromatic percarboxylic acid.

2. Process according to claim 1, in which the reaction is performed at a temperature of from −100 to +150° C.

3. Process according to claim 1, in which the reaction is performed at a pressure of from 0.5 to 100 bar.

4. Process according to claim 1, in which the reaction is performed in a reaction medium with a content of imidoaromatic percarboxylic acid of from 0.1% to 90% by weight relative to the total weight of the reaction medium.

5. Process according to claim 1, in which gradual or continuous addition of the imidoaromatic percarboxylic acid is performed.

6. Process according to claim 1, in which the imidoaromatic percarboxylic acid is introduced into the reaction in the form of a solution in an organic solvent or in pure form.

7. Process according to claim 1, in which the reaction is performed for a time of from 10 minutes to 50 hours.

8. Process according to claim 1, in which the reaction is performed in the substantial absence of water.

9. Process according to claim 1, in which the organic precursor is chosen from substituted or unsubstituted cyclic or acyclic olefins, substituted or unsubstituted cyclic or acyclic ketones, compounds containing at least one oxidizable sulfur atom, compounds containing at least one oxidizable selenium atom, compounds containing at least one oxidizable nitrogen atom and compounds containing at least one oxidizable phosphorus atom.

10. Process according to claim 1, in which the organic precursor is an oxidizable fluoro compound.

11. Process according to claim 1, in which the imidoaromatic percarboxylic acid is ε-phthalimidoperoxyhexanoic acid.
